(19) United States
(12) Plant Patent Application Publication
(10) Pub. No.: US 2012/0304348 P1
(43) Pub. Date: Nov. 29, 2012

(54) Scaevola humilis plant named 'PFS100'

(50) ;

(76) Inventor: Peter Abell, Bilpin (AU)

(21) Appl. No.: 13/506,937

(22) Filed: May 25, 2012

Related U.S. Application Data
(60) Provisional application No. 61/519,691, filed on May 27, 2011.

(51) Int. Cl. A01H 5/00

(52) U.S. Cl. PLT/363

(57) ABSTRACT

‘PFS100’ is a distinctive variety of Scaevola humilis, which is characterized by its combination of prostrate growth habit, large leaves, and stability and uniformity of traits through successive cycles of asexual propagation.

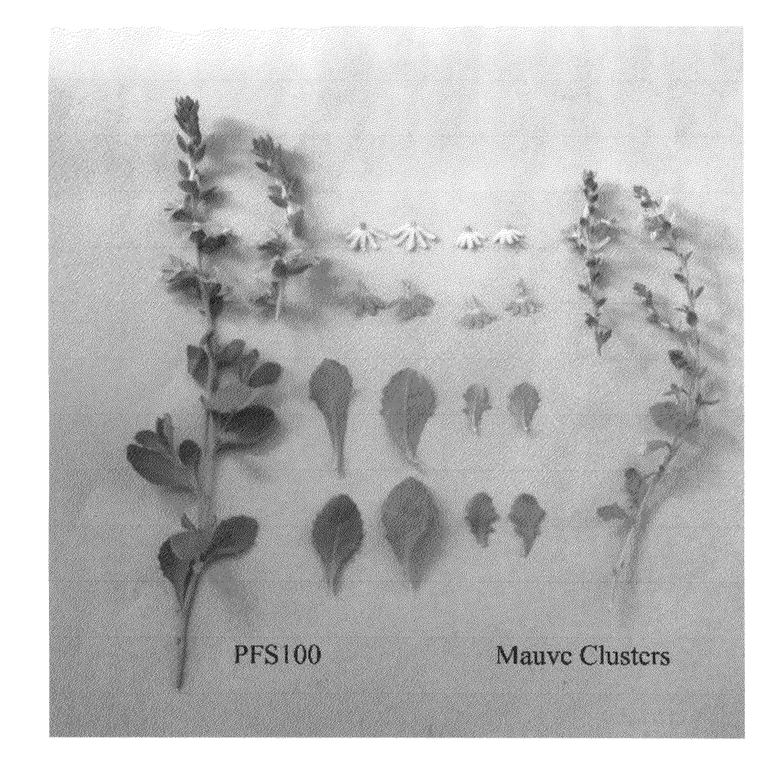

The present application claims priority from a provisional U.S. application No. U.S. 61/519,691 filed May 27 2011, which is herein incorporated by reference.

### LATIN NAME OF THE GENUS AND SPECIES

The Latin name of the genus and species of the novel variety disclosed herein is Scaevola humilis.

### VARIETY DENOMINATION

The inventive variety of Scaevola humilis disclosed herein has been given the variety denomination ‘PFS100’.

### BACKGROUND OF THE INVENTION

The present invention relates to a new and distinct perennial variety of Scaevola humilis, which has been given the variety denomination of ‘PFS100’. Its market class is that of an ornamental plant. ‘PFS100’ is intended for use in landscaping as a groundcover or potted plant.

An application for plant breeders' rights for variety ‘PFS100’ has been lodged with the Australian Plant Breeders' Rights Office, and was first gazetted in 14 Dec. 2010 under Application No. 2010/229.
Parentage: The Scaevola humilis variety ‘PFS100’ was selected in 2010 in an Australian nursery in the state of New South Whales following a selection process carried out from 2005 to 2010. ‘PFS100’ is the result of open pollination of Scaevola humilis (unpatented) whereby a resulting seedling was selected in March 2005 due to its flat growth habit. In June 2005, cuttings were taken from this selection for further evaluation. From August 2005 to January 2008, three subsequent cutting generations were propagated to increase the Inventor's trial sample for further evaluation. In 2008, rooted cuttings were transferred to 200 mm pots for further observation. In 2010, additional cuttings were taken and rooted. From these plants, rooted cuttings were transferred to 200 mm pots for evaluation and confirmation of distinctness, uniformity and stability (DUS). ‘PFS100’ was finally selected in April 2010 due to its flat growth habit and large leaves.
Asexual Reproduction: ‘PFS100’ is propagated asexually by division. The distinctive characteristics of the inventive ‘PFS100’ variety are stable from generation to generation; clones of the variety produced by asexual reproduction maintain the distinguishing characteristics of the original plant.

### SUMMARY OF THE INVENTION

‘PFS100’ is a distinctive variety of Scaevola humilis which is characterized by the combination of prostrate growth habit and large leaves.

As a groundcover form of Scaevola, ‘PFS100’ has a flat growth habit with an overall plant height of approximately 3 cm (unpruned), a width of approximately 25 cm (unpruned) and strong shoot density. ‘PFS100’ has a medium to long leaf length (40 to 55 mm) and a medium to broad width (20 to 25 mm).

### BRIEF DESCRIPTION OF THE FIGURES

FIGURE shows a 1 ‘PFS100’ plant (left) with comparator ‘Mauve Clusters’ (right) showing differences in leaf and flowers characteristics.

### BOTANICAL DESCRIPTION OF THE PLANT

The following is a detailed botanical description of a new and distinct variety of Scaevola humilis known as ‘PFS100’ based upon observations of 10-week old plants grown from rooted cuttings into 200 mm nursery pots in full sun in protected tunnel houses covered with plastic in Clarendon, New South Wales, Australia during spring 2010-summer 2010. Plant observations and descriptions were taken in summer 2010.

Those skilled in the art will appreciate that certain characteristics will vary with older or, conversely, with younger plants. ‘PFS100’ has not been observed under all possible environmental conditions. Where dimensions, sizes, colors and other characteristics are given, it is to be understood that such characteristics are approximations or averages set forth as accurately as practicable. The phenotype of the variety may differ from the descriptions set forth herein with variations in environmental, climactic and cultural conditions. Color notations are based on The Royal Horticultural Society Colour Chart, The Royal Horticultural Society, London, 2007 edition.

In southern climates, corresponding approximately to USDA Hardiness Zones 9 through 11, ‘PFS100’ is an evergreen perennial Scaevola humilis. ‘PFS100’ is a prostrate-growing groundcover plant, with long and broad leaf blades and a dense growth habit.

A botanical description of ‘PFS100’and comparisons with other varieties of Scaevola humilis are provided below.
Technical description of the variety.
   Plant characteristics.—Growth habit prostrate, height very short (average foliar height in a 200 mm pot 3 cm), narrow width (average width 25 cm) shoot density strong.
   Stems.—Strong branching habit, branch angles acute. Shape is cylindrical with a smooth texture, green in color (approximately RHS 146B).
   Leaves.—The simple leaves are petiolate with very short petioles, arrangement is alternate. Shape is ovate, margins shallow and dentate, apex acute, base cuneate, venation reticulate. Color of the upper leaf surface is green corresponding to RHS 137B and lower leaf surface corresponding to RHS 146B. Leaf texture in all stages is soft and glabrous to finely pubescence. Upper leaf surface exhibits strong glossiness and lower surface, medium glossiness. Mature leaves length medium with an observed length of 40 to 55 mm (average 46.8 mm) and observed width of 20 to 26 mm (average 24mm).
   Flowering.—‘PFS100’ displays a bloom habit similar to the seed parent: non-fragrant purple fan-shaped flowers with a very small eye. Flowers are not persistent, lasting approximately 7 days with 10 to 12 flowers per branch. ‘PFS100’ flowers in spring, summer and fall.
   Buds.—Shape is oblong and lanceolate. Approximate length of 12 mm and a width of 3 mm. Green in color (approximately RHS 146B) with underlying hues of purple (approximates to N82B).
   Perianth.—Zygomorphic, flat and fan-shaped, consisting of five basally-attached petals arising from the leaf axils. Observed width across the fan measures 20 to 24 mm (average width 21.4 mm). Observed length of the petals from point of attachment measures 10 to 12 mm (average length 10.8). Predominant color is violet, corresponding to approximately RHS N87C.
   Petals.—Five in number; shape is lanceolate; apex is cuspidate; margin is entire. Texture is smooth and glabrous. Color of both the upper and lower petal surfaces is violet (RHS N87C).
   Sepals.—Consists of one large ovate sepal and two smaller ligulate sepals with sessile attachment at the base of the flower. Apices are acute and margins are entire. Texture for all is lightly pubescent. Color is green, corresponding to approximately RHS 146B.
   Cold and heat tolerance.—‘PFS100’ has cold and heat tolerance typical of Scaevola humilis.
   Drought tolerance.—‘PFS100’ has good drought tolerance typical of Scaevola humilis once established.
   Pest resistance.—No known insect pests. ‘PFS100’ has disease tolerance typical of Scaevola humilis.
   Cultural conditions.—‘PFS100’ is tolerant of a wide range of soil types and pH ranges.
Comparisons with other scaevola: ‘PFS100’ was identified by the Royal Botanic Gardens Sydney Herbarium as Scaevola humilis. The only variety of Scaevola humilis known to the Inventor that closely matches ‘PFS100’ is Scaevola humilis ‘Mauve Clusters’. ‘PFS100’ is characterized by a flat, prostrate growth habit with an overall plant height of approximately 3 cm in a 200 mm pot (unpruned) at approximately 10 weeks of age, whereas ‘Mauve Clusters’ plants of similar age will reach a height of approximately 5 to 6 cm. At this age, ‘PFS100’ plants will display a very broad width of approximately 25 cm (unpruned) as compared with ‘Mauve Clusters’ which has a medium width of approximately 18 to 20 cm. ‘PFS100’ has a medium to long leaf length measuring 40 to 55 mm (midway between base of plant and first flowering node), whereas Scaevola humilis ‘Mauve Clusters’ has a short leaf length of approximately 20 to 25 mm. At an average width of 24 mm, the leaves of ‘PFS100’ are considered medium to broad for the species, whereas the leaves of Scaevola humilis ‘Mauve Clusters’ are considered narrow, measuring approximately 10 to 15 mm. The shallow, dentate leaf margins of ‘PFS100’ contrast to the medium to deeply sinuate leaf margins of ‘Mauve Clusters’. The lower leaf surface of ‘PFS100’ is slightly darker green (RHS 146B) when compared to ‘Mauve Clusters’ (RHS 144A). The lower leaf surface of ‘PFS100’ has medium glossiness whereas the lower leaf surface of ‘Mauve Clusters’ has slight glossiness. The flowers of ‘PFS100’ display a deeper violet color in comparison with those of ‘Mauve Clusters’ primarily due to the fact that the lower petal surfaces of ‘PFS100’ are more deeply purpled (RHS N87C) compared to those of ‘Mauve Clusters’ which correspond to RHS 85B.

That which is claimed is:

1. A new and distinct variety of Scaevola humilis plant named ‘PFS100’, substantially as described and illustrated herein.
